(19) United States
(12) Patent Application Publication
(10) Pub. No.: US 2013/0090516 A1
(43) Pub. Date: Apr. 11, 2013

(54) Local Anesthesia By Magnet-Directed Concentration of Nanoparticle Conjugated Anesthetic

(71) Applicants: UNVERSITY OF PITTSBURGH - OF THE COMME, Pittsburgh, PA (US); CARNEGIE MELLON UNIVERSITY, Pittsburgh, PA (US)

(72) Inventors: Vankat Ramana Rao MANTHA, Swissvale, PA (US); Krzysztof A. Matyjaszewski, Pittsburgh, PA (US)

(73) Assignees: CARNEGIE MELLON UNIVERSITY, Pittsburgh, PA (US); UNIVERSITY OF PITTSBURGH - OF THE COMMONWEALTH SYSTEM OF HIGHER EDUCATION, Pittsburgh, PA (US)

(21) Appl. No.: 13/645,904

(22) Filed: Oct. 5, 2012

Related U.S. Application Data
(60) Provisional application No. 61/626,913, filed on Oct. 5, 2011.

(51) Int. Cl. A61K 31/295; A61N 2/10; A61P 23/02; A61K 31/555; B82Y 5/00; B82B 1/00

(52) U.S. Cl. 600/12; 514/502; 514/188; 514/186; 977/773; 977/906; 977/811; 977/838

(57) ABSTRACT

Methods and formulations for induction of local anesthetic effects employing magnetic nanoparticles conjugated to anesthetic molecules. Magnetic nanoparticle-local anesthetic conjugates may be safely injected intravenously into human and animal subjects without encountering the deleterious effects observed with traditional injections of local anesthetics. The magnetic nanoparticle-local anesthetic conjugate may be concentrated at a site of action through the application of an external magnetic field to the patient at a site where local anesthesia is desired.

### CROSS-REFERENCE TO RELATED APPLICATION

This application claims the benefit of the earlier filing date of U.S. Provisional Application Serial Number 61/626,913 filed on Oct. 5, 2011.

### GOVERNMENT FUNDING

This invention was made with government support under grant numbers 0549353 and 0304568 awarded by the National Science Foundation. The government has certain rights in the invention.

### BACKGROUND OF THE INVENTION

1. Field of the invention

This invention relates generally to the field of anesthesiology, and more specifically to formulations for the intravenous administration of magnetic nanoparticle conjugated anesthetics and their use in inducing local anesthetic effects.

2. Description of the background

Local anesthesia is an integral tool for medical professionals for the treatment of human and animal subjects. Local anesthetics reduce or eliminate the tactile and pain sensations blocking transmission in pain fibers and allow the medical professional to manipulate anesthetized tissue without fear of causing the patient pain. Unlike general anesthesia, local anesthesia allows the patient to be awake and aware during the procedure, thus promoting patient confidence and well-being during the medical procedure. Additionally, local anesthesia avoids the some of the risks associated with general anesthesia, such as nausea, vomiting, and malignant hyperthermia.

The application of nerve and plexus blocks in humans, to the ankle as well as other locations around the body, provides a common example of local anesthetic use. The purpose of the block is to produce anesthesia in the part of the body supplied by the blocked nerves. Generally, such blocks involve injecting local anesthetic drugs into tissue close to nerve bundles. The anesthetic drug infiltrates the nerve and blocks action potentials through reversible inhibition of voltage-dependent sodium channels, and as a result blocks nociception. The vast majority of the nerves and/or nerve bundles are in close proximity to major arteries and veins. Accidental injection of local anesthetic into the blood vessels can have severe effects and may potentially result in immediate convulsions and cardiorespiratory arrest. Thus, great care is taken to avoid direct injection of the anesthetic into circulatory system.

Due to these concerns, nerve blocks are performed by medical doctors who have been highly trained in their administration. Nonetheless, the current practice is to perform these blocks either blindly using a nerve stimulator or under ultrasound guidance. Because of the potentially severe effects and the relative dearth of guidance during the procedure, the medical professional performing the technique needs to receive considerable training and possess substantial skill.

Many compounds are available for use as local anesthetics. Local anesthetics may be divided into two broad categories based on their chemical structure. Amide-containing anesthetics include articaine, bupivacaine, cinchocaine, etidocaine, levobupivacaine, lidocaine, mepivacaine, prilocaine, ropivacaine, and trimecaine. In particular, ropivacaine is a commonly employed and preferred local anesthetic that displays somewhat reduced cardiotoxicity compared to other previously used anesthetics. Another class of local anesthetics contains an ester chemical residue and includes benzocaine, chloroprocaine, cocaine, cyclomethycaine, dimethocaine, piperocaine, propoxycaine, procaine, proparacaine, and tetracaine.

The medical field possesses a long-standing need of allowing the facile and effective administration of local anesthetic drugs, while at the same time reducing the potentially catastrophic side effects of their use or misadministration. The present invention satisfies those needs.

### SUMMARY OF THE INVENTION

The present invention provides methods and formulations for induction of local anesthesia. The formulations may include magnetic nanoparticles conjugated to anesthetic molecules that are able to be safely injected into subjects without encountering the deleterious effects observed with traditional injections of local anesthetics. The magnetic nanoparticle-local anesthetic conjugate may be concentrated at a site of action through the application of an external magnet to the patient at a site where local anesthesia is desired.

The present invention provides formulations that include nanoparticles made up of a polymer and magnetic component that are conjugated with an anesthetic. In some embodiments, the polymer is poly(diethylene glycol)methyl ether methacrylate. The magnetic component may be a ferromagnetic component that may include nickel, iron, cobalt, gadolinium, a mixture thereof, an alloy thereof, or salts thereof. In some embodiments, the ferromagnetic component is ferric oxide (magnetite).

The anesthetic portion of the conjugate may be either an ester- or amide- containing local anesthetic. The amide-containing local anesthetic may be any one of articaine, bupivacaine, cinchocaine, etidocaine, levobupivacaine, lidocaine, mepivacaine, prilocaine, ropivacaine, trimecaine, pharmaceutically acceptable salts thereof, and combinations thereof, with ropivacaine hydrochloride being particularly useful in the context of the present invention.

If an ester-containing local anesthetic is used, it may be any one of benzocaine, chloroprocaine, cocaine, cyclomethycaine, dimethocaine, piperocaine, propoxycaine, procaine, proparacaine, tetracaine, pharmaceutically acceptable salts thereof, and combinations thereof.

The present invention provides for the induction of local anesthesia in a patient in need thereof by undertaking the following steps. First, a polymeric nanogel may be formulated through atom transfer radical polymerization and then magnetic nanoparticles are incorporated into the nanogel to a final concentration by weight from about 1% to about 25% by weight of the nanoparticle. Following the conjugation of the local anesthetic and the magnetic nanoparticles, their subsequent isolation, an aqueous suspension may be formed. The aqueous suspension may be intravenously injected into the patient and an external magnet is applied to an exterior of said patient at a site where local anesthesia is desired. The external magnet may take numerous geometric configurations, including a sleeve, a ring, a cuff, or an arc.

Prior to administration to the patient, the aqueous suspension may be kept stable at a temperature ranging from about zero degrees Celsius to about twenty degrees Celsius.

### BRIEF DESCRIPTION OF THE DRAWINGS

For the present invention to be clearly understood and readily practiced, the present invention will be described in conjunction with the following figure, which is incorporated into and constitute a part of the specification, wherein:

FIG. 1 shows a graph of paw withdrawal latency for control and experimental conditions.

### DETAILED DESCRIPTION OF THE INVENTION

It is to be understood that the figure and descriptions of the present invention have been simplified to illustrate elements that are relevant for a clear understanding of the invention, while eliminating for purposes of clarity, other elements that may be well known.

The present invention provides methods and formulations for inducing local anesthetic effects using magnetic nanoparticles (MNPs) conjugated to molecules of anesthetic. As noted above, the misadministration of local anesthetics is accompanied by potentially fatal medical complications. The present invention provides for a solution to such issues by allowing the concentration of local anesthetic molecules at the preferred cite of anesthesia by a mechanism distinct from direct injection into the surrounding tissue. The present invention provides for the conjugation of local anesthetic molecules to magnetic nanoparticles that are on the order of approximately 15 nanometers. The conjugated anesthetic-nanoparticle complex may be injected intravenously with minimal deleterious effects. It is currently thought that because of the conjugated nature of the local anesthetic in the formulation, the severe medical complications of systemic administration are avoided.

The magnetic nanoparticles circulating in the blood stream may be localized to the desired site of action through an external magnet applied to the skin of human or animal patient. While not wishing to be bound to theory, it is hypothesized that upon concentration at that site, the anesthetic molecule dissociates from the magnetic particle and is able to achieve the desired pharmacological anesthetic effect.

The formulations of the present invention may be generated in the following manner. Initially, a nanogel is formed from an appropriate polymer according to prior art techniques. In certain embodiments, the nanogels measure between about 200 and about 300 nanometers in depth. In some embodiments, poly(diethylene glycol)methyl ether methacrylate is used to form the nanogel. Additional polymers that may be employed in the context of the present invention include ethylene glycol, isoglycerol methacrylate, 2-(hydroxyethyl) methacrylate, hydroxypropyl methacrylate, eoligo(ethylene glycol) methyl ether methacrylate (OEOMA₄₇₅), vinyl acetate, N-vinyl acetamide, N-vinylpyrolidone, N,N-dimethylaminoethyl methacrylate, N-(hydroxyethyl)acrylamide, N,N-dimethylacrylamide, N-isopropylacrylamide, N-(2-hydroxypropyl) methacrylamide, vinylcarbonates, vinylcarbamates, 2-methacryloyloxyethyl phosphorylcholine, 3-methacryloxypropyl trimethoxysilane, exemplified by POEO₃₀₀MA-co-PHEMA nanogels, poly(ethylene glycol) monomethyl ether methacrylate, copolymers comprising poly(lactide), poly(lactide/glycolide) or poly(lactic acid/glycolic acid) segments, sulfobetaine methacrylate, and oligo(ethylene oxide) methacrylate. Additionally, functional groups of any of the above-named polymers may optionally be reacted with additional functional groups and provide polymers useful for the formulation of nanogels and conjugation with the selected anesthetic.

Nanogels useful within the context of the present invention may be synthesized by a controlled radical polymerization (CRP) of biocompatible comonomers, optionally including divinyl monomers, in a miniemulsion or microemulsion process. CRP procedures include atom transfer radical polymerization (ATRP) as disclosed in U.S. Pat. No. 8,273,823, or as reversible addition-fragmentation chain transfer (RAFT) as disclosed in U.S. Pat. No. 7,132,491, and/or nitroxide mediated radical polymerization (NMRP) as disclosed in U.S. Patent App. Pub. No. 2008/0038650, all of which are hereby incorporated by reference. A discussion of ATRP polymerization, including specific consideration of ATRP in miniemulsion may be found in K. Min & K. Matyjaszewski, “Atom Transfer Radical Polymerization in Aqueous Dispersed Media,” Cent. Eur. J. Chem. 7(4):657-674 (2009), which is hereby incorporated by reference. Generally, the nanogel may be prepared by heterogeneous polymerization of monomers in the presence of either difunctional or multifunctional cross-linkers. Activators may be generated by electron transfer ATRP to produce nanogels having a narrow size distribution, uniform crosslinking, and favorable physical properties. In some formulations, the polymers are biocompatible and biodegradable such that they break down in the body after administration to the patient.

To the formulated nanogel, magnetic nanoparticles may be added. The magnetic nanoparticles may be employed at a wide variety of sizes. The magnetic nanoparticles may range from about 5 nanometers to about 35 nanometers. In certain embodiments, the magnetic nanoparticles may range from about 10 nanometers to about 20 nanometers. In certain embodiments of the present invention, magnetic nanoparticles having a size of approximately 15 nanometers are used. The magnetic particles may be physically incorporated into the polymer nanogel during formation of the polymer. The concentration of magnetic particles incorporated into the nanogel may vary widely from about 1% by weight to about 25% by weight, with certain embodiments including at about 12 percent by weight of the nanogels. An example of the synthesis of the magnetic nanoparticles and subsequent incorporation into nanogels may be found in H. Dong, V. Mantha, & K. Matyjaszewski, “Thermally Responsive PM(EO)₂MA Magnetic Microgels via Activators Generated by Electron Transfer Atom Transfer Radical Polymerization in Miniemulsion,” Chem. Mater. 21:3965-3972 (2009), which is hereby incorporated by reference.

The nanoparticles may be made magnetic by including a ferromagnetic component, including nickel, iron, cobalt, gadolinium, mixtures thereof, and alloys thereof. In some embodiments, the magnetic nanoparticles include ferric oxide (magnetite) as the ferromagnetic component.

To provide the formulation with anesthetic properties, a local anesthetic may be physically loaded into the magnetic nanogels by mixing nanogels with an aqueous solution of a local anesthetic. The mixture may be kept a low temperature (e.g., 4° C.) for an extended period of time (e.g., from about 24 hours to about 72 hours). During this incubation, the local anesthetic associates with the magnetic nanoparticles and forms a magnetic nanoparticle-local anesthetic conjugate. As used herein, “conjugate” and “conjugation” include any chemical or physical association between the magnetic nanoparticle and the local anesthetic. While not wishing to be bound by theory, conjugate includes passive physical incorporation and chemical incorporation (ionic and/or covalent bonding).

Prior to use in patients, the conjugates may be purified from the mixture through any standard purification process such as centrifugation. Once purified the drug-loaded magnetic nanogel particles may be suspended in water. Generally, the resulting suspension is stable in water at temperatures ranging from about 0° C. to about 20° C.

While described particularly with regards to the local anesthetic ropivacaine, the present invention is equally applicable to a wide variety of both amide- and ester-based compounds. Amide-containing anesthetics useful within the context of the present invention include articaine, bupivacaine, cinchocaine, etidocaine, levobupivacaine, lidocaine, mepivacaine, prilocaine, ropivacaine, and trimecaine. Ester-containing local anesthetics useful within the context of the present invention include benzocaine, chloroprocaine, cocaine, cyclomethycaine, dimethocaine, piperocaine, propoxycaine, procaine, proparacaine, and tetracaine. Non-local anesthetics (e.g., propofol) may also be used within the context of the present invention. In short, any anesthetic compound that may be conjugated to a magnetic nanoparticle may be employed in the manner described herein. Within the context of the present invention, the local anesthetic may be utilized as a pharmaceutically acceptable salt, such as a hydrochloride, citrate, and tartarate.

Once formulated and injected, the magnetic nanoparticles-anesthetic complex may be concentrated at the desired location using an external magnetic field. The specific configuration of the external magnet may be widely varied with the geometry of the body part to be anesthetized dictating some geometric considerations. For the blockade of ankles or wrists, a sleeve configuration may be employed, while for non-extremities a more planar disk configuration may be preferable. One of skill in the art will recognize the appropriate configuration to be utilized based on the particular medical application confronting the user.

The following procedure provides a presently preferred example, though commonly known deviations from the procedure below, including variations in the polymeric basis of the nanogel, concentration of components (e.g., local anesthetic, magnetic particles), and methods for isolation of nanoparticles.

### EXAMPLE 1

Poly(diethylene glycol)methyl ether methacrylate (PM(EO)₂MA) nanogels measuring 200-300 nanometers were synthesized by ATRP in miniemulsion. Nanogels were produced in the following manner. CuBr₂(3.0 mg, 0.013 mmol), bis(2-pyridylmethyl) octadecylamine (6.1 mg, 0.013 mmol), M(EO)₂MA (2.0 g, 10.6 mmol), and anisole (0.6 mL) were charged into a round-bottom flask. The resulting mixture was stirred at 65° C. for at least 2 hours to dissolve the copper complex and then cooled to room temperature. The ethyl 2-bromoisobutyrate initiator (4.0 μL 0.027 mmol), bis(2-methacryloyloxyethyl) disulfide cross-linker (15.7 mg, 0.054 mmol), and hexadecane (95 μL) were added into the cold solution. Aqueous polyoxyethylene(20)oleyl ether solution (10.32 mL, 5 mmol/L) was added to the organic solution before the mixture was subjected to sonication (output control set at 8 and duty cycle at 70% for 2.5 minutes). The resulting homogenized miniemulsion was transferred to a Schlenk flask and purged with nitrogen for 50 minutes. The flask was then immersed in an oil bath thermostatted at 65° C. An aqueous solution of hydrazine (0.5 mL, 8.13 μmol/mL) was injected into the reaction to initiate the polymerization. After 19.0 hours, the reaction was stopped by opening the flask and exposing the catalyst to air. The monomer conversion was 75.3%, determined by gravimetry. The cross-linked nanogels were purified by consecutive centrifugation (10,000 rpm for 20 minutes) and redispersed in tetrahydrofuran (THF). Finally, the nanogels in THF were subjected to solvent exchange treatment to obtain a toluene suspension.

Magnetic ferric oxide (Fe₃O₄) nanoparticles modified with oleic acid having a size of approximately 15 nanometers were synthesized in the following manner. Ferrous sulfate heptahydrate (FeSO₄·7H₂O, 2.35 g, 8.45 mmol) and ferric chloride hexahydrate (FeCl₃·6H₂O, 4.10 g) were added to a 100 mL flask. The flask was sealed with a rubber septum and purged with nitrogen for 1 hour. About 100 mL of N₂-bubbled deionized water was then injected into the flask to dissolve the salts. The solution was vigorously stirred, followed by addition of ammonium hydroxide solution (25 mL, 28-30 wt %) quickly at room temperature. The solution color changed from orange to black, indicating formation of Fe₃O₄ precipitates. Oleic acid (“OA”; 1.5 mL) was then slowly injected under vigorous stirring into the dispersion at 80° C. over 1 hour. The whole process was carried out under a nitrogen atmosphere. After that, the Fe₃O₄/OA nanoparticle water dispersion was mixed with toluene (100 mL). By adding a small amount of sodium chloride, Fe₃O₄/OA nanoparticles transferred into the toluene phase. Finally, the toluene dispersion was refluxed to remove most of the water under the nitrogen atmosphere, and the concentration of Fe₃O₄/OA was diluted with toluene to 17 mg/mL.

The magnetic nanoparticles thus produced were physically incorporated into the polymer nanogel at about 12% by weight of the nanogel in the following manner. The PM(EO)₂MA nanogel toluene suspension (10.0 g, 17 mg/mL) was mixed with the Fe₃O₄/OA nanoparticle toluene suspension (10.0 g, 17 mg/mL) for 48 hours at room temperature. The PM(EO)₂MA nanogels loaded with Fe₃O₄/OA nanoparticles were separated from free Fe₃O₄/OA nanoparticles by consecutive centrifugation (10,000 rpm for 20 minutes) and redispersed in toluene until the supernatant was colorless. The nanogels were black and well dispersed in toluene. To disperse the magnetic nanogels into water, toluene was first exchanged to THF by centrifugation, and then the nanogel-THF dispersion was slowly dropped into a large amount of cold water in ice bath. The magnetic nanogels were finally precipitated by heating the water to 40° C. and collected by magnet. The precipitate was washed by 40° C. water several times to remove THF, redispersed into 0° C. water, and stored at 4° C.

Ropivacaine hydrochloride was physically loaded into the magnetic nanogels by mixing magnetic nanogels with 1% solution of ropivacaine hydrochloride in 4 milliliters of water. The mixture was kept at 4° C. for 48 hours. The mixture was purified on the day of use by removing free drug via centrifugation (10,000 RPM for 20 minutes) and washing three times with water at 4° C. The final aqueous solution included 8 mg/ml of nanogel containing 12% by weight of ferric oxide (magnetite) nanoparticles and 7 mg/ml of ropivacaine. The drug-loaded magnetic nanogel was stably suspended in water at 20° C. Prior to administration to subjects, the suspension was kept between 5 and 10° C.

Conjugated nanoparticles so prepared may be injected intravenously into a subject. The conjugated nanoparticles do not have significant toxic effects on subjects when prepared in accord with the present invention. The efficacy as a local anesthetic when concentrated with an external magnet was assessed in the following experiment.

### EXAMPLE 2

Sprague Dawley rats (300-350 grams) were used to assess the efficacy of formulations of ropivacaine generated as described above. The Hargreaves test of thermal nociception evaluates the level of sensory anesthesia of the plantar surface of the paw and was used to assess pain sensation here. Animals were placed in individual Plexiglas testing chambers in a modified Hargreaves box. Hargreaves et al., 1988, which is hereby incorporated by reference. A very low power idle intensity beam was used to initially target the plantar surface of the hind paw. Once appropriately placed, the beam was switched to a higher power active intensity (approximately 50% of maximum intensity). This causes radiant heat stimulation of the plantar hind paw and allows for a measure of the latency of the rat's paw withdrawal threshold.

Before each experiment, baseline nociception testing was performed on the animals. In all experiments, the left paws were used as controls and the right paws were utilized in the treatment condition. Rats were anesthetized using the general anesthetic isoflurane, awaked after a designated time, and the paws were tested after their returned to baseline wakefulness (approximately 5-10 minutes). Each hind paw was tested at 5 minute intervals for 40 minutes and at 10-minute intervals for 120 minutes. A baseline-, pre-anesthesia paw withdrawal latency was set at 10-12 seconds, with a cutoff at 20 seconds to prevent tissue injury.

As a control condition, a traditional ankle block was performed by administering 0.8 ml of 0.1% or 0.2% ropivacaine subcutaneously. Rats were immediately awakened after injection and sensory testing was performed.

In a test condition, a dose of magnetic nanoparticle-ropivacaine was injected intravenously into a tail vein of the rat. Magnetic sleeves were placed around the right hind paw of the rat. In the present experiment, several magnet configurations were employed—disks, arcs, and rings. Disk sleeves included three disk magnets ( 7/16″× 3/16″) and cloth tape. Arc magnets (¾″×¾″×⅛″) were attached to either a cardboard loop or a strip magnet. The ring magnet configuration included soft, pliable plastic tubing containing a ring magnet (1″ outer diameter, ½″ inner diameter, and ¼″ thick) and a small disk magnet immediately below it. In the data presented below a ring configuration of magnets was used.

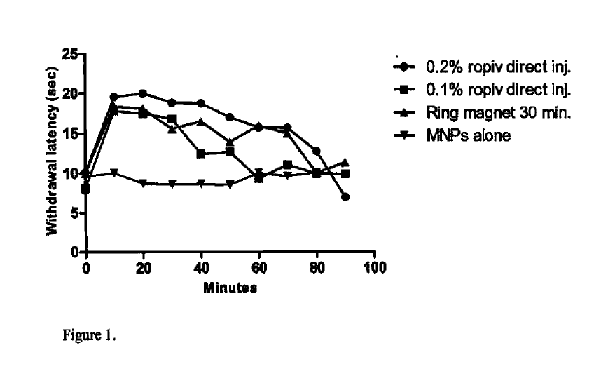

Representative results are shown in FIG. 1. In the data shown, a ring magnet was applied for thirty minutes in 11 animals prior to testing to allow for concentration of magnetic nanoparticles-local anesthetic complexes at the site of testing. The response latency in the rat was significantly prolonged compared to baseline (P<0.01) when the magnetic nanoparticle-ropivacaine conjugate was injected. As expected, a traditional nerve block resulting from subcutaneous injection of ropivacaine (either 0.1% or 0.2%)%), in six animals each, was achieved. Further, when the animal was injected with only magnetic nanoparticles (n=6), there was no change in the withdrawal latency. The left paw showed no change from baseline withdrawal latency in any experiment.

### EXAMPLE 3

While the results of Example 2 demonstrate that formulations of magnetic nanoparticles-ropivacaine may cause local anesthesia, additional experiments were performed to assess safety of the formulation on test animals. Specifically, acute toxicity studies of unconjugated magnetic nanoparticles, assay of plasma concentration of ropivacaine, and measurement of tissue concentration of ropivacaine at the ankle were performed.

The experiments were done after placing the animals under isoflurane anesthesia and spontaneous ventilation. A volume of 2 milliliters of the injectate was given at 5-15° C. intravenously via a tail vein cannula over four to five minutes. A jugular catheter was inserted where necessary for blood withdrawals.

Acute toxicity studies of unconjugated magnetic nanoparticles were conducted in 10 animals. Cardiopulmonary arrest was taken as evidence of toxicity. No immediate or delayed (24 hours) toxicity was observed.

For the assessment of plasma concentration of ropivacaine, the following groups were studied:

| | |
|---|---|
| Group Aₚₗₐₛₘₐ: | ropivacaine only. |
| Group Bₚₗₐₛₘₐ: | MNP/ropivacaine, no magnet. |
| Group Cₚₗₐₛₘₐ: | MNP/ropivacaine + magnet 30 minutes. |

Each group included 3 animals. A volume of 0.3 ml of blood was drawn before injection, and at 15, 30, 60, 120, 180, 240 and 300 minutes after the injections. In group Aₚₗₐₛₘₐ, 1 mg of ropivacaine was used because a slightly higher dose (1.2 mg) proved lethal. The animals in groups Bₚₗₐₛₘₐ and Cₚₗₐₛₘₐ had 14 mg of ropivacaine complexed with MNPs. In group Cₚₗₐₛₘₐ, the magnet assembly used in Example 2 was placed around the right ankle before injection and removed after 30 minutes.

After intravenous administration, the plasma concentration of ropivacaine declined in an exponential manner as expected, in all three groups. The various pharmacokinetic parameters are given in Table 1. Group Aₚₗₐₛₘₐ cannot be directly compared to the other two groups because the close in group Aₚₗₐₛₘₐ was 14 times lower. Between groups Bₚₗₐₛₘₐ and Cₚₗₐₛₘₐ, there were no differences in any parameter. Data are presented as mean (SD). Lambda-Z is defined as terminal elimination rate constant. AUC is the area under the concentration curve. V_{d} is the volume of distribution. Cl is clearance rate. N/A means not applicable. Listed probability values are established through t-tests between groups B and C.

| | | | | | Group C | | |
|---|---|---|---|---|---|---|---|
| | Group A | | Group B | | MNP/ropiv + | | |
| | Plain ropiv | | MNP/ropiv | | magnet | | Prob. |
| Lambda-Z | 0.44 | (1.10) | 0.54 | (0.14) | 0.52 | (0.25) | 0.91 |
| Cₘₐₓ (ng/ml) | 863.3 | (761) | 115.5 | (97.9) | 165.9 | (129.6) | 0.63 |
| Cₘₐₓ | 863.3 | (751) | 8.25 | (6.99) | 11.85 | (9.26) | 0.08 |
| ng/ml/mg) | | | | | | | |
| (dose | | | | | | | |
| normalized) | | | | | | | |
| Tₘₐₓ (h) | 0.25 | | 0.25 | | 0.25 | | N/A |
| Half life (h) | 1.63 | (0.42) | 1.37 | (0.38) | 1.60 | (0.89) | 0.72 |
| AUC | 875.0 | (589.2) | 115.8 | (64.1) | 225.4 | (197.7) | 0.46 |
| (0-inf, | | | | | | | |
| ng/ml/h) | | | | | | | |
| AUC | 875.0 | (589.2) | 8.27 | (4.6) | 16.1 | (14.1) | 0.46 |
| ng/ml/h, mg) | | | | | | | |
| (dose | | | | | | | |
| normalized) | | | | | | | |
| V_{d} (ml) | 3180 | (1099) | 269559 | (95110) | 174237 | (51957) | 0.23 |
| Cl (ml/h) | 1462 | (715) | 151076 | (86481) | 98607 | (66784) | 0.47 |

A separate set of two animals for each condition were used to assess ropivacaine concentration in the ankle. The groups were named as below.

| | |
|---|---|
| Group Aₜᵢₛₛᵤₑ: | ropivacaine only. |
| Group Bₜᵢₛₛᵤₑ: | MNP/ropivacaine, no magnet. |
| Group Cₜᵢₛₛᵤₑ: | MNP/ropivacaine + magnet 30 minutes. |

In all groups, 0.3 ml of blood was withdrawn at baseline, at 15 minutes, and at 30 minutes for plasma ropivacaine determination. At 30 minutes, skin and subcutaneous tissue all around the right ankle were isolated for ropivacaine analysis. Analysis was performed by HPLC-MS-MS.

The tissue drug concentration was much higher in group Group Aₜᵢₛₛᵤₑ compared to the other groups; however, the ratio of tissue to plasma concentration at 30 minutes displayed a trend towards being lower, as shown in Table 2. Between Group Bₜᵢₛₛᵤₑ and Cₜᵢₛₛᵤₑ the absolute tissue concentration and the tissue to plasma concentration ratio displayed a trend towards being higher in the magnet group. Again, data are presented as mean (SD)

| | | Tissue conc. | Plasma conc. | |
|---|---|---|---|---|
| Group | Time (h) | (ng/g) | (ng/ml) | Ratio |
| A | 0.5 | 1050 (110) | 336.5 (13.7) | 3.12 |
| B | 0.5 | 105 (20) | 25.2 (1.2) | 4.17 |
| C | 0.5 | 150 (10) | 24.9 (2.3) | 6.02 |

The doses in groups B and C were higher by a factor of 14 compared to group A but the plasma Cₘₐₓ values were lower by a factor of 5-7.5. This conforms to the existing literature, where the plasma levels of bupivacaine and ropivacaine were several times lower when formulated in liposomes compared to drug alone. See Yu HY, Li SD, Sun P. Kinetic and dynamic studies of liposomal bupivacaine and bupivacaine solution after subcutaneous injection in rats. J Pharm Pharmacol. 2002; 54(9): 1221-7; and Shen Y, Ji Y, Xu S, Chen da Q, Tu J. Multivesicular liposome formulations for the sustained delivery of ropivacaine hydrochloride: preparation, characterization, and pharmacokinetics. Drug Deliv. 2011; 18(5): 361-6. This result may also explain the observed safety of the ropivacaine when combined with MNPs.

The absolute tissue drug concentration at the ankle and the tissue to plasma concentration ratio were higher in the magnet group (group C) than in group B, as would be expected. However, the absolute tissue drug concentration in group A was higher by a factor of 7-10 compared to groups B and C.

In conclusion, the present pharmacological studies demonstrated that, when conjugated to MNPs, ropivacaine was safe in very high doses. The tissue-to-plasma concentration of ropivacaine tended to be highest in the magnet group, showing successful sequestration and concentration of ropivacaine at the ankle by the magnet.

Nothing in the above and attached descriptions is meant to limit the present invention to any specific materials, geometry, or orientation of elements. Many modifications are contemplated within the scope of the present invention and will be apparent to those skilled in the art. The embodiments disclosed herein were presented by way of example only and should not be used to limit the scope of the invention.

What is claimed is:

1. A formulation for the induction of local anesthesia, comprising:
nanoparticles, comprising a polymer and magnetic component; and
an anesthetic, wherein said nanoparticles and said anesthetic form a conjugate.

2. The formulation of claim 1, wherein said magnetic component is a ferromagnetic component.

3. The formulation of claim 2, wherein said ferromagnetic component includes nickel, iron, cobalt, gadolinium, a mixture thereof, an alloy thereof, or salts thereof.

4. The formulation of claim 3, wherein said ferromagnetic component is ferric oxide.

5. The formulation of claim 1, wherein said anesthetic is a local anesthetic.

6. The formulation of claim 6, wherein said local anesthetic is selected from the group consisting of an amide-containing local anesthetic and a ester-containing local anesthetic.

7. The formulation of claim 6, wherein said amide-containing local anesthetic is selected from the group consisting of articaine, bupivacaine, cinchocaine, etidocaine, levobupivacaine, lidocaine, mepivacaine, prilocaine, ropivacaine, trimecaine, and pharmaceutically acceptable salts thereof.

8. The formulation of claim 6, wherein said amide-containing local anesthetic is ropivacaine.

9. The formulation of claim 8, wherein said ropivacaine is ropivacaine hydrochloride.

10. The formulation of claim 6, wherein said ester-containing local anesthetic is selected from the group consisting of benzocaine, chloroprocaine, cocaine, cyclomethycaine, dimethocaine, piperocaine, propoxycaine, procaine, proparacaine, tetracaine, and pharmaceutically acceptable salts thereof.

11. The formulation of claim 1, wherein said polymer is a biocompatible copolymer.

12. The formulation of claim 11, wherein said biocompatible copolymer is selected from the group consisting of poly(lactide), poly(lactide/glycolide) or poly(lactic acid/glycolic acid) segments, sulfobetaine methacrylate, and oligo(ethylene oxide) methacrylate.

13. The formulation of claim 11, wherein said biocompatible polymer is poly(diethylene glycol)methyl ether methacrylate.

14. A method of inducing local anesthesia in a patient in need thereof, comprising the steps of:
formulating a polymeric nanogel;
incorporating magnetic nanoparticles into said nanogel;
conjugating a local anesthetic with said magnetic nanoparticles to form magnetic nanoparticle-local anesthetic conjugates;
isolating said magnetic nanoparticle-local anesthetic conjugates;
suspending said magnetic nanoparticle-local anesthetic conjugates in water to form an aqueous suspension;
intravenously injecting said aqueous suspension of said magnetic nanoparticle-local anesthetic conjugates into a patient in need thereof; and
applying an external magnet to an exterior of said patient at a site where local anesthesia is desired.

15. The method of claim 14, wherein said polymeric nanogel is formed by controlled radical polymerization.

16. The method of claim 15, wherein said controlled radical polymerization is atom transfer radical polymerization.

17. The method of claim 14, wherein said magnetic nanoparticles possess a diameter between about 2 nanometers and about 35 nanometers.

18. The method of claim 17, wherein said magnetic nanoparticles possess a diameter between about 5 nanometers and about 24 nanometers.

19. The method of claim 18, wherein said magnetic nanoparticles possess a diameter of approximately 15 nanometers.

20. The method of claim 14, wherein said magnetic nanoparticles are present in a concentration from about 1% by weight to about 25% by weight of the nanogel.

21. The method of claim 14, wherein said magnetic nanoparticles include nickel, iron, cobalt, gadolinium, a mixture thereof, an alloy thereof, or salts thereof.

22. The method of claim 14, wherein said aqueous suspension of said magnetic nanoparticle-local anesthetic conjugates are maintained from about 0 degrees Celsius to about 20 degrees Celsius prior to said intravenously injecting step.

23. The method of claim 14, wherein said local anesthetic is selected from the group consisting of an amide-containing local anesthetic and a ester-containing local anesthetic.

24. The method of claim 23, wherein said amide-containing local anesthetic is selected from the group consisting of articaine, bupivacaine, cinchocaine, etidocaine, levobupivacaine, lidocaine, mepivacaine, prilocaine, ropivacaine, trimecaine, and pharmaceutically acceptable salts thereof.

25. The method of claim 24, wherein said amide-containing local anesthetic is ropivacaine.

26. The method of claim 25, wherein said ropivacaine is ropivacaine hydrochloride.

27. The method of claim 23, wherein said ester-containing local anesthetic is selected from the group consisting of benzocaine, chloroprocaine, cocaine, cyclomethycaine, dimethocaine, piperocaine, propoxycaine, procaine, proparacaine, tetracaine, and pharmaceutically acceptable salts thereof.

28. The method of claim 14, wherein said external magnet is present as a sleeve, a ring, a cuff, or an arc.
